(19) United States
(12) Patent Application Publication
(10) Pub. No.: US 2007/0198050 A1
(43) Pub. Date: Aug. 23, 2007

(54) Medical implant device

(75) Inventors: Adrian Ravenscroft, Rochester, MA (US); Yimin Yang, Medfield, MA (US)

Correspondence Address: NIXON PEABODY, LLP, 401 9TH STREET, NW, SUITE 900, WASHINGTON, DC, 20004-2128, US

(73) Assignee: Phase One Medica, LLC, Carver, MA (US)

(21) Appl. No.: 11/543,247

(22) Filed: Oct. 5, 2006

Related U.S. Application Data
(60) Provisional application No. 60/775,355, filed on Feb. 22, 2006.

(51) Int. Cl. A61M 29/00

(52) U.S. Cl. 606/200

(57) ABSTRACT

An improved implant device is deployed a vessel, or passageway, in a body with an attachment mechanism that permits stable and secure positioning of an implant device while also permitting easy removal without damaging the vessel. In particular, clip-like structures are used to engage the wall of the passageway. A tether that works in cooperation with the attachment mechanism may be used to facilitate removal of the implant device after an indicated period. The tether is removable from the implant device to convert the implant device into another configuration, which can remain permanently or be removed at a later time. Moreover, a centering mechanism may be employed to ensure that the implant device is properly oriented within the passageway when deployed.

### CROSS REFERENCE TO RELATED APPLICATIONS

This application claims priority to U.S. Provisional Application No. 60/775,355 filed Feb. 22, 2006, the contents of which are incorporated herein by reference.

### BACKGROUND OF THE INVENTION

1. Field of the Invention

The present invention generally relates to the deployment of a medical implant device in a body passageway, and more particularly to the deployment of medical implant devices with an attachment mechanism which removably attaches the implant device to the wall of the body passageway.

2. Description of Related Art

Venous Thromboembolic Disease (VTE) is a disease where a blood clot forms in a blood vessel and blocks the blood flow. In particular, major surgery or severe trauma can increase the risk of VTE because blood clots are more likely to form. In some cases, the clot travels to other sites in the body, an occurrence known as an embolism. VTE includes deep venous thrombosis (DVT) and pulmonary embolism (PE). DVT involves clots sitting deep within the veins, where they interfere with the blood flow. PE involves a clot that has traveled to the lung, where it may cause death in a short period of time if it goes untreated. VTE is the third most common cardiovascular disease and a leading cause of death in the United States. An estimated 300,000 patients are hospitalized each year for treatment of acute DVT. It is estimated that an additional 1.2 million people suffer from undiagnosed DVT. It is also estimated that DVT affects 20% to 30% of all major surgical patients. The most feared complication of DVT is PE. Currently, PE is the third leading acute cardiovascular cause of death in the United States. PE is reported in up to 650,000 patients a year with an estimated mortality of 240,000 per year. The highest recognized incidence of PE occurs in hospitalized patients, with 60% of hospitalized patients having had a PE. However, the diagnosis is missed in 70% of those patients.

Anticoagulation therapy has been the recommended treatment for patients suffering from DVT and PE. When anticoagulation cannot be used or when it fails, placement of a permanent vena cava filter in the inferior vena cava has been indicated. Vena cava filters have been used since 1973 as the alternative therapy to anticoagulation.

In the last five years, a growing trend toward more aggressive prophylactic management of DVT and/or PE has developed. It began with development of a first generation of optional/retrievable vena cava filters, which gave physicians the ability to place filters in patients and have the option of removing them at a later date or leaving them permanently in place. This has opened and broadened indications for placement of filters to a larger category of patents whom, due to severe trauma or major surgery, experience a one-time risk of DVT and/or PE. Younger patients who require short-term prophylaxis are of particular clinical interest. Once these patients are ambulatory, they are at low risk of developing DVT and/or PE and do not require caval interruption. Therefore, a temporary/optional filter is a good alternative to permanent caval interruption with a vena cava filter.

In addition to trauma and major surgery, two new procedures are beginning to be indicated for prophylactic placement of optional vena cava filters. Obese patients undergoing weight loss surgery (Bariatric) are at substantial risk for developing thromboembolic disease. PE is a leading cause of death following bariatric surgery for morbid obesity. To date, only 0.6% of an estimated 11.5 million morbidly obese patients have had bariatric surgery. Bariatric procedures have grown from 13,365 in 1998 to an estimated 130,000 in 2005. Procedure growth to 218,000 in 2010 is projected. Bariatric surgery is expected to become the standard of care for obesity. The increasingly important role of surgical therapy for treating the morbidly obese has brought more relevance to the issue of prophylaxis for PE in this patient population. Those patients considered to be at high risk for DVT/PE are candidates for prophylactic filter placement. Roles for IVC filter placement in this population are expanding as more data is acquired.

Prophylactic placement of a retrievable filter for PE prevention during percutaneous mechanical thrombectomy (PMT) and/or thrombolytic therapy in lower limb or acute iliofemoral DVT is now regarded as a clinical indication. Although anticoagulation is effective in preventing PE, many patients go on to experience post-thrombotic syndrome, which is a chronic sequelae of DVT, with resultant valvular insufficiency of the lower extremity. Endovascular management using PMT and/or thrombolysis has recently received much attention in the literature as a safe and effective means for the treatment of acute DVT. The efficacy of IVC filter placement during acute DVT management using endovascular techniques has been reported in several clinical reports. In one study, 132 patients with lower-extremity DVT were implanted with retrievable filters prior to thrombolytic therapy. Study authors reported the presence of thrombus in the filters after thrombolytic therapy in 41 (31%) of the 132 patients. This type of improvement in medical therapy to treat DVT has heightened awareness of primary care physicians regarding the clinical sequelae of DVT and the need to treat more aggressively. It is estimated that up to 160,000 patients are candidates for PMT with another 150,000 patients candidates for thrombolytic therapy. Currently, only an estimated 20,000 are treated with an endovascular intervention.

The vena cava filters described provide one example of the variety of medical devices that have been recently developed for permanent or temporary implantation in the human body. Despite recent advancements, however, achieving effective deployment and positioning of such devices remains a challenge. Moreover, even when a device has been successfully implanted, keeping the implanted device in the desired position for an extended period of time presents an even greater challenge. Devices that are specifically designed for temporary implantation are often difficult to keep in position, because the methods by which they are held in place are purposely weak in order to permit subsequent removal.

A number of such medical implant devices are designed to collapse for insertion within a catheter or other delivery unit and to expand to a predetermined shape when ejected after delivery. Many of these self expanding devices rely primarily upon the contact between the device and the wall of a body vessel or passageway to maintain the device in position after the delivery unit is removed. Unfortunately, changes in the dimensions of the body vessel or passageway or variations in the flow or pressure of blood or other fluids therethrough can cause the medical implant to migrate and change position.

In an attempt to prevent migration of a medical implant device, rigid hooks are often formed on the device to engage the wall of a body vessel or passageway as the implant device expands into contact with the wall. After a few weeks, the endothelium layer grows over the rigid hooks which will not easily bend under the influence of withdrawal pressure, and the medical implant device will be locked in place by the embedded hooks. Locking an implant device in place with embedded hooks may be acceptable for a permanent implant, but rigid hooks are not a viable option if the medical implant device is to be removed after several weeks or months.

To facilitate removal of a previously implanted medical device by withdrawal of the hooks from an enveloping endothelium layer without risking substantial damage to the wall of a body vessel or passageway, the hooks have been formed to straighten when subjected to a withdrawal force greater than a maximum migration force.

The use of hooks to prevent migration of an implanted medical device can be subject to a number of disadvantages. In previous devices, the hooks are engaged due to the expansion of the device into contact with the wall of a body vessel or passageway. The engagement of the hooks is caused only as a result of the expansion of the device and is not a function which is separable from such expansion. Thus, there can be instances where one or more hooks fail to properly engage the wall of a body vessel or passageway.

To alleviate many of the problems experienced with hooks, medical device anchor and delivery systems have been developed which do not anchor a medical implant device to the wall of a vessel or passageway until after the device has fully expanded into contact with a first surface of the wall. Once expansion has been completed, each anchor is ejected from a delivery tube surrounding the anchor and is propelled through the wall of the vessel or passageway from the first side to a second side where the anchor expands against the second side. The expanded anchor is formed to straighten in response to a withdrawal force to permit the anchor to be withdrawn from the wall of the vessel or passageway. These systems, disadvantageously, require the use of a delivery tube for each anchor and a delivery mechanism for holding the medical implant device in position while ejecting each anchor from a delivery tube and positively propelling the anchor through the wall of the vessel or passageway.

As indicated previously, implant devices are often only required for a temporary period. In particular, patients who are at a one-time temporary risk of pulmonary embolism should receive a vena cava filter, but it is often clinically difficult to justify placement of a permanent filter due to the associated long-term complications. Thus, temporary devices are designed to be removed after short-term residence. Such devices are typically removed by an elective interventional procedure by ensnaring the filter tip with a capturing cone or snare system. In some cases, however, the filter cannot be removed without causing significant vessel wall injury, if the filter has become tilted or the tip becomes incorporated into tissue.

Attempts have been made to employ tethers to facilitate removal of temporary implant devices. Such tethers, extending through a passageway, generally have a proximal end accessible from outside the body and a distal end attached to the implant device. However, such devices have been largely unsuccessful. Tethered filters have failed clinically and commercially for a variety of adverse events. Such systems suffer from an adverse event known as migration, which is caused by buckling of the tether when the device becomes loaded with blood clots. Rather than hooks, barbs or anchors, some tethered systems rely on the column strength of the tether to prevent filter migration. A second adverse event, known as duration of indicated use, is created by logistical and clinical complications associated with venous thromboembolic disease. If PE occurs and the temporary filter traps a clot, the clot is likely to remain in the filter at the time the device is supposed to be removed. Because the tether remains attached to the device, the device cannot be converted into a permanent filter and left in place. This inability to convert the filter to a permanent implant after the temporary indication period presents a serious dilemma.

### SUMMARY OF THE INVENTION

The present invention provides secure attachment and proper orientation of an implant device in a passageway in a body, while also facilitating removal of the implant device without causing damage to the passageway. As such, the present invention overcomes the shortcomings of, and provides advantages over, the known techniques described above. For instance, with the attachment mechanism of the present invention, the implant device can advantageously be used with a removable tether to permit conversion of the implant device from a temporary to an optional implant device, which may remain in the passageway indefinitely or be removed at a later time.

Accordingly, an exemplary embodiment of the present invention is illustrated by an implant device adapted to be removably attached to a wall of a body passageway. The implant device includes a clip with a space for receiving at least a part of the wall of the passageway. The space is formed by a contact portion and a clip leg. The clip leg has a sharp tip for engaging the wall of the passageway. The clip leg is positioned on the contact portion and extends away from the contact portion at an angle. The contact portion and the clip leg resist an increase in the space between the contact portion and the clip leg. Thus, the part of the wall received in the space is clamped between the contact portion and the clip leg, thereby attaching the implant device to the wall of the passageway.

In a particular embodiment, the implant device has a longitudinal axis and a plurality of elongate legs extending away from the longitudinal axis at an angle. The implant device may be a blood clot filter with a clot-capturing basket formed by the plurality of elongate legs. Each of the plurality of elongate legs has a contact portion. A clip leg is positioned at the contact portion of each of the plurality of elongate legs while extending away from the contact portion at an angle. The implant device is attached to the wall of the passageway by moving the implant device along the passageway in a first direction and moving the clip leg into engagement with the wall of the passageway, causing a part of the wall to be received and held in the space between the body and the leg clip. On the other hand, the implant device is detached from the wall of the passageway by moving the implant device along the passageway in a second direction opposite the first direction.

The plurality of elongate legs on the exemplary implant device above may be collapsible for guiding the implant device to a position in the passageway, and expandable for removable attachment to the wall of the passageway. In particular, the implant is positionable within a retractable sheath. The retractable sheath containing the implant device can then be guided to a position in the passageway, while the sheath keeps the implant device collapsed during movement through the passageway. The sheath can then be retracted from the implant device to allow the implant device to expand into attachment with the wall of the passageway at the location.

A control mechanism may be employed to control positioning of the implant device within the passageway. The control mechanism may include an elongate tube with a distal end and a proximal end, the tube having a plurality of longitudinal slits at the distal end of the tube. A control wire passes through the elongate tube and is operable from the proximal end of the tube. A nodule is connected to the control wire at the distal end of the tube and moves with the operation of the control wire to engage the longitudinal slits of the tube. The control mechanism engages the implant device when the longitudinal slits at the distal end of the tube are passed through an aperture in the implant device and the nodule engages the longitudinal slits, causing the tube to expand outwardly at the longitudinal slits to an expanded width greater than the aperture width, so that the tube cannot pass back through the aperture. The control mechanism is releasable from the implant device when the longitudinal slits of the tube are free from engagement by the nodule and the tube has a non-expanded width less than the aperture width, allowing the tube section to pass through the aperture.

A tether, with a distal end and a proximal end, may be attached to the implant device at the distal end and may be operable at the proximal end to move or position the implant device. An extension wire may be attached to the proximal end of the tether to extend the tether and to enable a sheath to be guided over the implant device. The extension wire may have a protrusion at the end of the extension wire that is capable of engaging a slot at the proximal end of the tether.

In particular, the tether is attached to an implant device which has an attachment mechanism, such as the clips above, to attach the implant device to a wall in the passageway. The implant device has an aperture for receiving a release mechanism. The tether has a distal end and a proximal end, where the proximal end is operable to control the implant device. A release mechanism releasably connects the tether to the attachment portion of the implant device. The release mechanism may operate similarly to the control mechanism described previously. Thus, the tether is detachable from the implant device.

In addition, a centering mechanism may be operably attached to the implant device proximate to a centered part of the implant device which should be kept near the center of the passageway. The centering mechanism has extensions extending outwardly from the centered part of the implant device. The extensions contact the interior surface of the wall of the passageway to space the wall away from the centered part. The implant device is guided with the centering mechanism to a location in the passageway. The implant device is then attached to the wall with the attachment mechanism while the centering mechanism keeps the centered part of the implant device near the center of the passageway. The centering mechanism may be directly attached to the implant device or may be indirectly connected, for instance, through a tether which is used to deploy the implant device.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates an implant device that employs tissue clips to removably attach the implant device to the wall of a blood vessel according to an aspect of the present invention.

FIG. 2 illustrates a contact portion and a tissue clip that can be employed by the implant device of FIG. 1.

FIG. 3 illustrates an embodiment of a control mechanism that can be employed to affix the position of the implant device of FIG. 1 during deployment.

FIG. 4 illustrates another embodiment of a tissue clip of the present invention.

FIG. 5 illustrates yet another embodiment of a tissue clip of the present invention.

FIG. 6 illustrates a further embodiment of a tissue clip of the present invention.

FIG. 7 illustrates another embodiment of an implant device that employs tissue clips according to an aspect of the present invention.

FIG. 8 illustrates an embodiment of an implant device that employs tissue clips and a centering mechanism according to aspects of the present invention.

FIG. 9 illustrates a further embodiment of an implant device that employs tissue clips and a centering mechanism according to aspects of the present invention.

FIG. 10 illustrates an embodiment of an implant device that employs a tether in addition to tissue clips according to aspects of the present invention.

FIG. 11 illustrates an embodiment of an extension wire which is attachable to the tether of the implant device of FIG. 10.

FIG. 12 illustrates an embodiment of an release mechanism that can removably attach the tether to the implant device of FIG. 10.

### DETAILED DESCRIPTION

The present invention overcomes shortcomings of the known devices described above by providing improved deployment of an implant device that is secured to a vessel, or passageway, in a body. The present invention provides an attachment mechanism that permits stable and secure positioning of an implant device while also permitting easy removal without damaging the passageway. The present invention also provides a tether that works in cooperation with the attachment mechanism to facilitate removal of the implant device after an indicated period. The tether is removable from the implant device to convert the implant device from a temporary device into an optional device, which can remain permanently or be removed at a later time. Moreover, the present invention provides a centering mechanism to ensure that the implant device is properly oriented within the passageway when deployed. These, and other, aspects of the present invention are provided in further detail below.

While the implant devices in the exemplary embodiments presented herein may be blood clot filters, it is understood that these embodiments are presented merely to demonstrate aspects of the present invention. Such aspects of the present invention are not limited to use with blood clot filters. In addition, the description provided herein may refer to the deployment of an implant device in a blood vessel in particular, but it is also understood that aspects of the present invention can be employed in any passageway in the body.

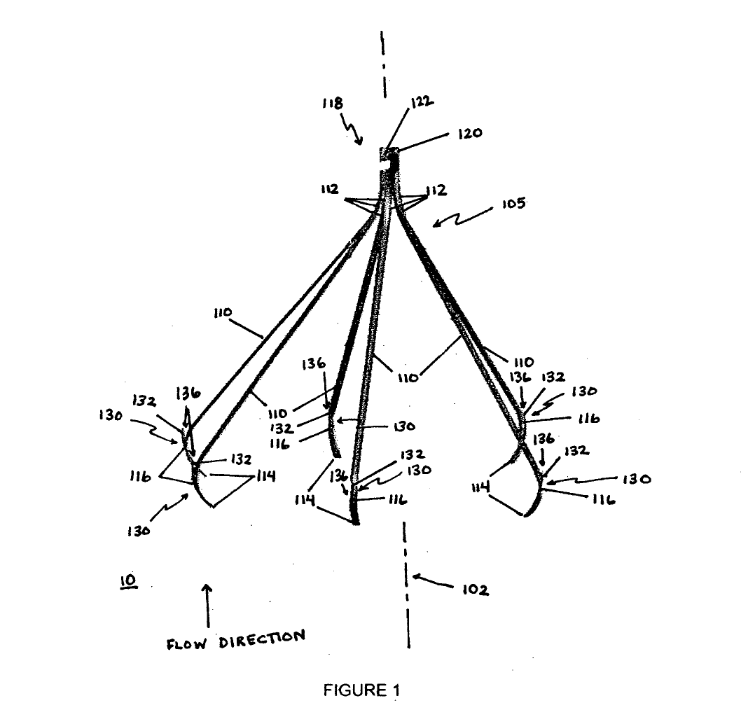

Accordingly, a blood clot filter 10 according to the present invention is illustrated in the exemplary embodiment of FIG. 1. The blood clot filter 10 is generally deployed within a blood vessel in order to trap blood clots that may form and travel within the blood vessel. The arrow shown in FIG. 1 depicts the flow direction within the blood vessel. As such, the filter 10 includes a plurality of elongate legs 110 which form a basket-like structure 105 that can collect blood clots that travel in the flow direction, into the interior of the basket-like shape. The filter 10 preferably has six or seven elongate legs, but may have any number legs 110 which are sufficient to create a clot-capturing basket 105.

The filter 10 has a central longitudinal axis 102, which is generally oriented with the elongate direction of the blood vessel when the filter is deployed. A filter cap 120 is positioned at an apex 118 of the filter 10, which is positioned on, or near, the central longitudinal axis 102. Each of the legs 110 has a connecting end 112 connected to the filter cap 120 to form the clot-capturing basket 105. The legs 110 are arranged evenly in a circular, or near circular, manner, around the central longitudinal axis 102. The legs 110 are proximate to each other at the filter cap 120, but spread apart as they extend generally in the upstream direction, i.e. against the flow direction. In other words, the legs 110 extend from the longitudinal axis 102 at an angle, as illustrated in FIG. 1. Each leg 110 also has a free curved end 114 opposite the connecting end 112. The curved end 114 curves toward the longitudinal axis 102, and thus, inwardly from the interior surface of the vessel wall when the filter 10 is deployed. In addition, each of the legs 110 has a contact portion 116 proximate to the curved end 114. The contact portion 116 makes contact with the interior surface of the wall of the blood vessel when the filter is deployed.

As further illustrated in FIG. 1, a clip 130 is formed by the contact portion 116 and a clip leg 132 positioned at the contact portion 116 of each leg. The clip leg 132 extends at an angle from the contact portion 116. In particular, it extends outwardly from the contact portion 116 at angle, generally in the flow direction. The clip leg 132 has a penetrating end 134 capable of penetration though a blood vessel wall. The clip leg 132 may be integral with the contact portion 116 or may be a separate component attached at contact portion 116.

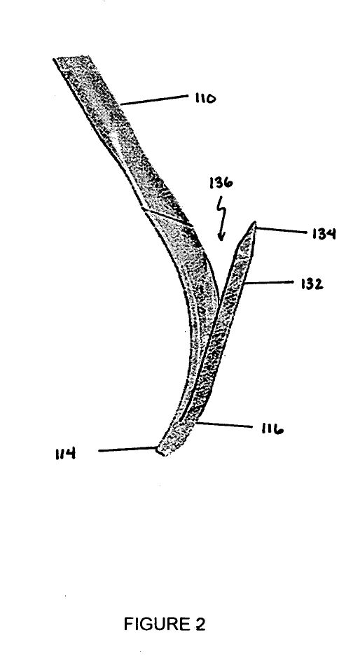

As shown in greater detail in the exemplary embodiment of FIG. 2, the contact portion 116 of the leg 110 is flattened. This flattened contact portion 116 is curved inwardly toward the central longitudinal axis 102 of the filter 10 and is designed to contact, and slide along, the inner surface of a blood vessel without penetrating the blood vessel surface. Secured to the contact portion 116 and angling outwardly away from the contact portion 116 is the clip leg 132. While the clip leg 132 may have a variety of shapes, the clip leg 132 shown in FIG. 2 is substantially a straight, non-curved leg which terminates with the penetrating end 134. While the flattened contact portion 116 and the leg 110 in general are flexible, the clip leg 132 is substantially rigid.

The legs 110 are formed of a flexible material which permits them to be flexibly bendable toward the central longitudinal axis 102 of the filter 10. Thus, the plurality of legs 110 is collapsible, or compressible, toward the longitudinal axis 102. It is preferable to form the elongate legs of a suitable shape memory material such as nitinol, although spring metal, suitable plastics, or other materials can be used to form the filter legs.

During deployment of the blood clot filter 10, the elongate legs 110 of the filter 10 are collapsed toward the longitudinal axis 102, making the filter 10 elongate in shape and allowing the filter 10 to fit within the elongate chamber, or channel, of a delivery tube, also known as an introducer sheath (not shown). The wall of the sheath keep the legs 110 collapsed. The filter 10, while remaining within the sheath, is delivered to a desired location within the blood vessel. If, for instance, the blood clot filter 10 is introduced into the vena cava from the jugular, the curved ends 114, pressed together, and lead the filter 10 as it is passed through the blood vessel. In other words, the curved ends 114 are positioned farther from the entrance through which the filter is introduced into the blood vessel, while the filter cap 120 remains positioned closer to this entrance. Once the filter 10 is position, it is then ejected, or moved, from the sheath to permit the legs 110 to expand outwardly to achieve the shape shown in FIG. 1 and into contact with the inner surface of the vessel wall via contact portion 116. The expansion of the legs 110 forms a full clot-capturing basket 105 that is open toward the flow direction.

Once the filter is positioned and released from the sheath, it is attached to the blood vessel wall by drawing it longitudinally in the flow direction to drive the substantially rigid clip legs 130 into, and preferably completely through, the vessel wall. The flat surface of the contact portion 116 slides on the inner surface of the vessel wall as the clip leg 132 penetrates, at least partially, the vessel wall with the penetrating end 134. When the clip leg 132 penetrates, or engages, the vessel wall, tissue from the vessel wall is forced into the space, or area, 136, between the leg 110 and the clip leg 132. This tissue is clamped by a biasing force exerted by the flexible and resilient leg 110 toward the rigid clip leg 132. In other words, the tissue received into the space 136 causes deflection of the leg 110 with respect to the clip leg 132, and with this deflection, the resilient leg 110 creates a biasing force which resists the expansion of the space 136. Thus, the tissue is clamped in the space 136 between the leg 110 and clip leg 132. To promote this clamping effect, the space 136 may be an acutely angled, or wedge-shaped, area. Therefore, the filter 10 is held securely and stably in place, and can resist any force that is less than a withdrawal force acting to draw the clip legs 132 out of the vessel wall.

In general, the blood clot filter 10 is attached to the wall of the blood vessel by moving the filter 10 in the flow direction to cause the clip legs 130 to engage the exterior surface of the wall and cause the wall to be held between the legs 110 and their respective clip legs 130. As noted, however, the clip leg 132 does not have to penetrate the vessel wall completely. Secure and stable positioning of the filter 10 can be achieved by moving the clip legs 132 into partial penetration, or engagement, with the vessel wall so that at least some tissue is held between the legs 110 and their respective clip legs 132.

To remove the blood clot filter 10, a force greater than the withdrawal force is applied generally opposite the flow direction, causing the rigid clip legs 132 to slide out of the vessel wall and release the clamped tissue from the area 136. Normally, the withdrawal force is applied to the clip legs 132 by pushing the filter cap 120 against the flow direction. The straight, rigid nature of each clip leg 132 and the flexibility of each leg 110 facilitate withdrawal and detachment of the filter 10 from the vessel wall while minimizing any damage to the wall. As the clip leg 132 is withdrawn, the flat contact portion 116 slides over the inner surface of the vessel wall with no penetration, the inward curve of the curved end 114 ensuring that the end of the leg 110 cannot penetrate the vessel wall during withdrawal.

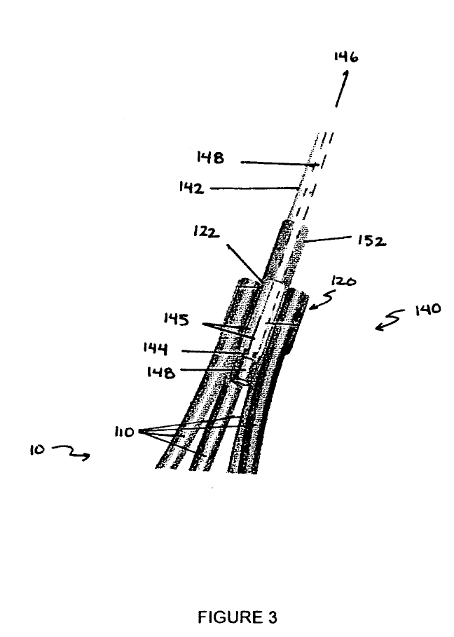

FIG. 3 illustrates a control mechanism 140 to hold the blood clot filter 10 during deployment and attachment to the blood vessel wall. The control mechanism is generally employed as a part of a filter delivery system. The control mechanism 140 includes an elongate tube 142 with a distal end 144 that engages the filter 10 and a proximal end 146 that corresponds with a proximal end of the delivery system which is accessible from outside the blood vessel and the body. A central control wire 148 passes through the elongate tube 142 and is operative from the proximal end of the filter delivery system. The central control wire 148 has an enlarged outer diameter at one end to form a nodule, or bulb-like structure, 149 which extends past the distal end 144 of the elongate tube 142 in the position shown. The distal end 144 is provided with spaced, longitudinally extending slits 145. The filter cap 120 has an aperture 122 with an aperture width. Both the distal end 144 and the nodule 149 are dimensioned to pass through the aperture 122 when the nodule 149 is positioned beyond the distal end 144 of the elongate tube 142. In this case, the distal end 144 and the nodule 149 each have a width less that the aperture width. A bushing, or stopping mechanism, 152 secured to the outer surface of the elongate tube 142 limits the distance that the elongate tube 142 can extend through the filter cap 120.

In the operation of the control mechanism 140, the blood clot filter 10 is attached to a filter delivery system by passing the elongate tube 142 and nodule 149 through the center aperture 122 in the filter cap 120 until the bushing 152 engages the filter cap 120. At this point, the slits 145 at the distal end 144 extend beyond the edge of the filter cap 120. When the control wire 148 is pulled back toward the proximal end of the delivery system, the nodule 149 moves into engagement with, and expands, the distal end 144 of the elongate tube 140 in the area of the slits 145. In other words, the control mechanism 140 engages the filter 10 at the filter cap 120 when the longitudinal slits 145 at the distal end 144 of the elongate tube 142 are passed through the aperture 122 and the nodule 149 engages the longitudinal slits 145. As a result, the tube 142 expands outwardly, or radially, at the longitudinal slits 145, to an expanded width greater than the width of the aperture 122. Once the distal end 144 of tube 142 is expanded, the filter cap 120 is then positioned, or held, between the bushing 152 and the expanded end 144. The control mechanism 140 can then be used to move the filter 10 longitudinally both in upstream and flow directions. The control wire 148 can be attached to a spring in a handle attached to the proximal end of a delivery system to hold the nodule in engagement with the distal end 144.

The elongate tube 142 may be formed of nitinol or similar material to facilitate this expansion while permitting the distal end 144 to return to the non-expanded width when the nodule 149 is removed. To release the filter 10 from the control mechanism 140, the control wire 148 is pushed forward to free the nodule 149 from engagement with the longitudinal slits 145 at the distal end 144. As a result, the elongate tube 142 at the area of the longitudinal slits 145 is allowed to return to its non-expanded width which is less than the width of the aperture. Accordingly, the nodule 149 and elongate tube 140 can be passed or withdrawn through the aperture 122 of the filter cap 120.

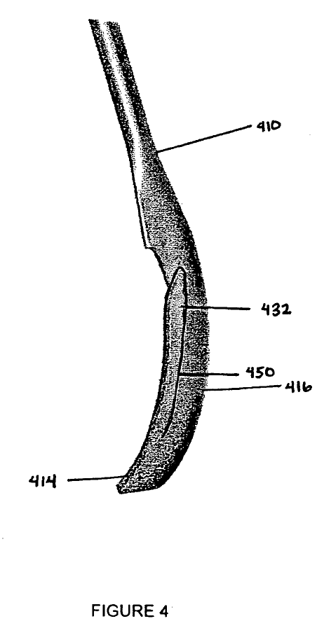

As further illustrated by FIGS. 4-6, the tissue clip of the present invention can be formed entirely from a flattened contact portion of an elongate leg. As FIG. 4 shows, each of the legs 410 of blood clot filter 40 is implemented to have a flattened contact portion 416. A slit 450 is cut through the flattened contact portion 416 in spaced relationship to one side edge of the contact portion 416 to form a pointed clip leg 432 which angles away from the contact portion 416 and extends away from the curved end 414 in the flow direction. The lower end of the slit 450 remains joined to the curved contact portion 416. As described previously, when the blood clot filter 40 is pulled longitudinally in the flow direction, the pointed clip leg 432 is driven through the blood vessel wall, and tissue is clamped between the substantially rigid clip leg 432 and the flexible contact portion 416, which applies a biasing force. The blood clot filter 40 is released from attachment with the blood vessel by pushing the filter 40 longitudinally against the flow direction to withdraw the clip leg 432 from the vessel wall and release the clamped tissue.

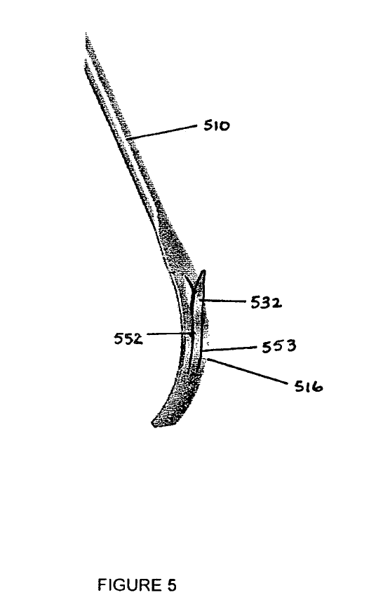

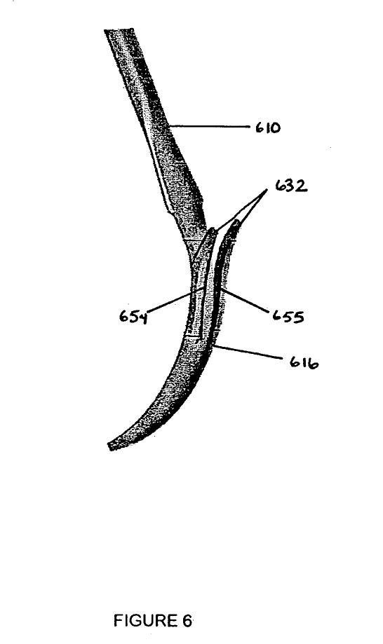

The tissue clips of FIGS. 5 and 6 are variations of the tissue clip of FIG. 4 and operate in the same manner to drive more rigid clip legs through a vessel wall when the blood clot filter is moved longitudinally in the flow direction, and tissue is clipped between the clip legs and the flat, more flexible contact portions. In FIG. 5, the clip leg 532 of blood clot filter 50 is formed centrally at the contact portion 516 by cutting two spaced slits 552 and 553 through the leg 510 at contact portion 516. Meanwhile, in FIG. 6, two spaced clip legs 632 of blood clot filter 60 are formed by cutting slots 654 and 655 spaced from the sides of the leg 610 at the contact portion 616.

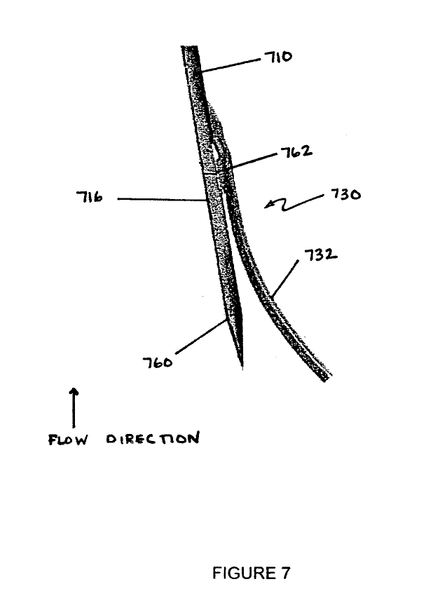

FIG. 7 shows a further embodiment of the present invention. The blood clot filter 70 has a tissue clip 730 with a contact portion 716 on an elongate leg 710 that is straight and substantially rigid and is formed with a point 760 at the distal end of the elongate leg 710. A flexible clip leg 732 is secured at one end to the contact area 716 and curves inwardly away from the contact portion 716. A resilient pad 762 may be provided between the contact portion 716 and the clip leg 732. The tissue clip 730 is designed to engage and clamp tissue from the blood vessel wall when the blood clot filter 70 is moved longitudinally against the flow direction. The contact portion 716 is inserted through a vessel wall, while the clip leg 732 slides over the inside surface of the vessel wall and tissue is clamped between the contact portion 716 and the clip leg 732. The tissue is released from the tissue clip 730 by pulling the blood clot filter 70 longitudinally in the flow direction to withdraw the rigid contact portion back through the vessel wall.

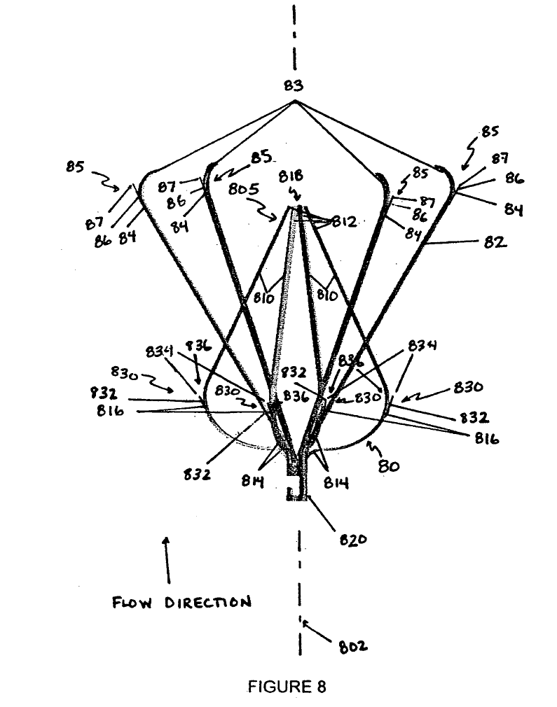

Referring to FIG. 8, another embodiment of the present invention is illustrated. The objectives of this embodiment include providing an implant device that is retrievable from the femoral vein, minimizes vessel injury upon removal, and minimizes non-centered deployment of the device. Accordingly, a blood clot filter 80 includes a clot-capturing basket 805 made of elongate basket legs 810 with capturing ends 812 which are adjacent to each other at a centerless apex 818. The filter 80 captures, or traps, a blood clot at the capturing ends 812. The capturing ends 812 are not joined to one another, but the basket legs 810 are stably and securely positioned to create a basket to filter blood clots traveling in the flow direction through the blood vessel. The filter 80 may have any number of basket legs 810 that is sufficient to create a clot-capturing basket 805, the capturing ends 812 being separated by a distance small enough to prevent blood clots from flowing through the clot-capturing basket 805. The filter 80 has a central longitudinal axis 802, which is generally oriented with the elongate direction of the blood vessel when the filter is deployed. The basket legs 810 are arranged evenly in a circular, or near circular, manner, around a central longitudinal axis 802. The basket legs 810 extend radially outward from the centerless apex 818 while also extending against the flow along the longitudinal axis 802, as illustrated in FIG. 8. The basket legs 810 have connected ends 814 opposite the capturing ends 812. The connected ends 814 curve inwardly toward the central longitudinal axis 802, so that the connected ends 814 connect at a filter cap 820. The filter cap 820 is adapted to accept a control mechanism 840, such as the control mechanism 140 above, for controlling the filter 80. The upstream position of the filter cap 820 permits control or retrieval of the filter 80 from an upstream position, such as the femoral vein. In addition, each of the basket legs 810 has a contact portion 816 which makes contact with the interior surface of the wall of the blood vessel when the filter is deployed.

As further illustrated in FIG. 8, a tissue clip 830 at each leg is formed by the contact portion 816 and a clip leg 832 positioned at the contact portion 816. The tissue clip 830 helps the filter 80 to achieve stability within the blood vessel. The clip leg 832 extends outwardly from the leg 810, while extending generally along the longitudinal axis 802 against the flow direction. Furthermore, the clip leg 832 has a penetrating end 834 capable of penetration into, or through, a blood vessel wall.

The filter 80 may be laser-cut from a single tube of a material such as nitinol. In particular, the basket legs 810 are sufficiently flexible to be collapsible for easier passage through the blood vessel. The filter 80 may be deployed through a low profile introducer sheath from either caudal or cephalic approaches. The sheath keeps the basket legs 810 collapsed in an elongate chamber, or channel, until the filter 80 reaches the desired position in the blood vessel. When the filter 80 emerges from the sheath, the material of the basket legs 810 allows the basket legs 810 to expand into contact with the vessel wall with the contact portions 816. Accordingly, the centerless apex 818 is positioned in order to trap a clot within the basket formed by the basket legs 810.

Once the filter 80 is positioned, the tissue clips 830 are employed to position the filter 80 stably and securely within the vessel wall. The tissue clips 830 can be attached to the vessel wall by pushing the filter in the flow direction from the femoral approach or pulling the filter 80 against the flow direction from the jugular approach, in order to achieve optimal migration resistance. In general, the filter 80 is attached to the wall of the blood vessel by moving the filter 80 along the vessel in the flow direction and moving the clip legs 832 into engagement with the wall of the passageway, causing parts of the wall to be received into a space 836 between the elongate basket legs 810 and the clip legs 832. This tissue is clamped by a biasing force exerted by the flexible and resilient leg 810 toward the rigid clip leg 832. In other words, the tissue received into the space 836 causes deflection of the leg 810 with respect to the clip leg 832, and with this deflection, the resilient leg 810 creates a biasing force which resists the expansion of the space 836. Thus, the tissue is clamped in the space 836 between the leg 810 and clip leg 832. To promote this clamping effect, the space 836 may be an acutely angled, or wedge-shaped, area.

The filter 80 is detachable from the wall of the blood vessel by movement of the filter 80 along the blood vessel against the flow direction. When the filter is positioned in the vena cava, the centerless apex 818 allows the filter 80 to be pulled from the vena cava with a snare or capturing cone from the femoral approach. Even if the filter 80 and its tissue clips 830 become incorporated into the tissue of the wall of the vessel, the filter 80 can be removed with minimal trauma. The radial strength of the basket legs 810 that make up the clot-capturing basket 805 are strong enough to capture clots but weak enough to allow for radial expansion/displacement during removal. In other words, the entire filter structure changes its shape to facilitate removal. For instance, the elongate legs 810 are able to straighten out for removal, due in part to the fact that the capturing ends 812 are not joined to one another.

A centering mechanism 82 is attached to the filter 80 which causes the filter, and more specifically the apex 818, to be centered within the wall of the blood vessel upon deployment. The centering mechanism ensures that the implant device is properly oriented within the passageway. In particular, the centering mechanism spaces a part of the implant device from the wall of the passageway to orient the implant device. Proper orientation of the implant device minimizes any chance that the filter will become tilted or that a tip of the filter will become incorporated into tissue. With reference to the embodiment of FIG. 8, the centering mechanism 82 is attached to the filter 80 which causes the filter, and more specifically the apex 818, to be centered within the wall of the blood vessel upon deployment. The centering mechanism 82 is positioned proximate to the connected ends 814, where the plurality of basket legs 810 are connected as described above. The centering mechanism includes a plurality of centering legs 83 which extend away from the longitudinal axis 802 of the filter 80 at an angle, generally in the flow direction. Each of the centering legs 83 contacts the wall with a contact portion 84. As shown in FIG. 8, the centerless apex 818 is positioned, or centered, between the contact portions 85. Thus, the centering legs 83 ensure that the centerless apex 818 remains spaced away from the blood vessel wall, so that the clot capturing basket 805 is in the position and orientation to capture clots traveling through the blood vessel. The centering mechanism 82 may have any number of centering legs 83 that is sufficient to center the centerless apex 818. Moreover, while FIG. 8 shows the centering legs 83 generally spaced evenly apart in a circular, or near circular, arrangement, the centering legs 83 of an implant device may be arranged differently, for instance, according to the passageway shape. The centering legs 83 are formed of a flexible material which permits them to be flexibly bendable toward the central toward the central longitudinal axis 802 of the filter 80, so that the filter 800 can be collapsed for deployment with the filter 80 in a sheath.

A tissue clip 85 may be formed by the contact portion 84 and a clip leg 86 angling away from the contact portion 84. The clip leg 86 extends generally in the flow direction and away from the contact portion 84 at an angle. Thus, the tissue clip 85 can receive at least a part of the wall of the body passageway. The centering leg 83 and the clip leg 86 resist movement of the clip leg 86 away from the centering leg 83 and to hold the tissue received between the contact portion 84 and clip leg 86. The clip leg 86 also includes a penetrating end 87 capable of penetration into the tissue of the wall of the passageway. Thus, filter 80 is further secured to the wall of the blood vessel with movement of the filter 80 in the flow direction as the clip legs 86 on the centering legs 83 penetrate at least a part of the wall and cause wall tissue to be held by the tissue clips 85.

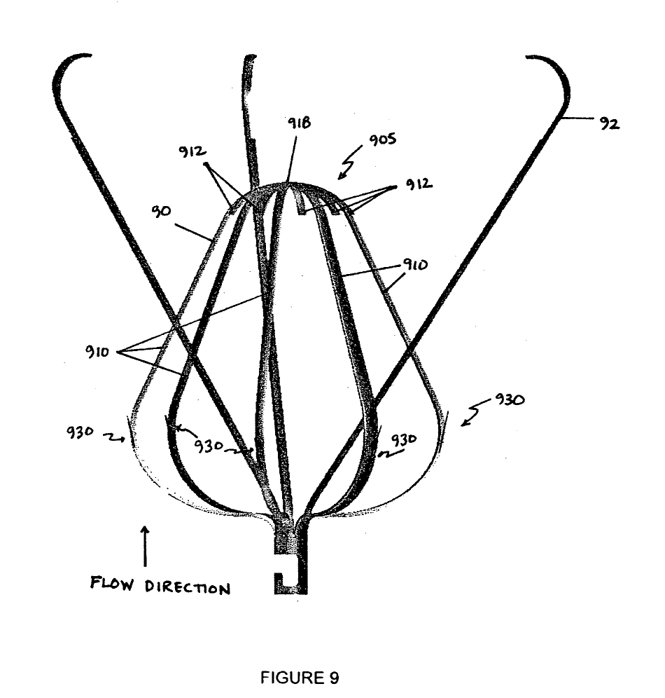

FIG. 9 illustrates another embodiment of the present invention. A blood clot filter 90 is similar to the filter 80 described previously. In particular, the filter 90 includes a clot-capturing basket 905 made of elongate legs 910 with capturing ends 912 at an apex 918 which are adjacent to one another but not joined to one another. Like the filter 80, the filter 90 may be deployed through a low profile introducer sheath from caudal and cephalic approaches. The tissue clips 930, similar to those of filter 80, can be attached to the vessel wall by pushing the filter in the flow direction from the femoral approach or pulling the filter 90 against the flow direction from the jugular approach, in order to achieve optimal migration resistance. In addition, a centering mechanism 92 is attached to the filter 90.

Furthermore, like the filter 80, the filter 90 is detachable from the wall of the blood vessel by movement of the filter 90 along the blood vessel against the flow direction. When the filter is positioned in the vena cava, the centerless apex 918 allows the filter 90 to be pulled from the vena cava with a snare or capturing cone from the femoral approach. Even if the filter 90 and its tissue clips 930 become incorporated into the tissue of the wall of the vessel, the filter 90 can be removed with minimal trauma. The radial strength of the basket legs 910 that make up the clot-capturing basket 905 are strong enough to capture clots but weak enough to allow for radial expansion/displacement during removal. In other words, the entire filter structure changes its shape to facilitate removal. For instance, the elongate legs 910 are able to straighten out for removal, due in part to the fact that the capturing ends. 912 are not joined to one another.

However, as illustrated in FIG. 9, the filter 90 is different from the filter 80, because the capturing ends 912 of elongate legs 910 curve to overlap each other, even though they remain unconnected. The crossing and curved legs 910 at the apex 918 provide additional stiffness to the capturing basket 905. Furthermore, the crossing legs 910 also provide additional filtering efficiency. During removal, the overlapping capturing ends 912 separate from one another and straighten out along with the elongate legs 910.

Although the embodiments of the present invention described with reference to FIGS. 1-9 employ a blood clot filter, the tissue clips, in particular, can be used to secure an implant device with any shape to the wall of a passageway in the patient's body. In general, the present invention has a body with a contact portion. The device is moved along the passageway and into contact against the interior surface of the wall of the passageway via the contact portion. A tissue clip is formed by the contact portions and a clip leg positioned at the contact portion, where the clip leg angles outwardly from the contact portion. In particular, the clip leg extends generally in the flow direction. The clip leg has an engaging end capable of engagement with the wall of the passageway. In operation, the implant device is attached to the wall of the passageway by moving the implant device along the passageway in the flow direction and moving the clip leg into engagement with the wall of the passageway, which causes a part of the wall to be held between the body and the leg clip. On the other hand, the implant device is detached from the wall of the passageway by moving the implant device along the passageway in the upstream direction to disengage the part of the wall from the tissue clip. Of course, the implant device can have more than one contact portion, each with a leg clip to form a tissue clip.

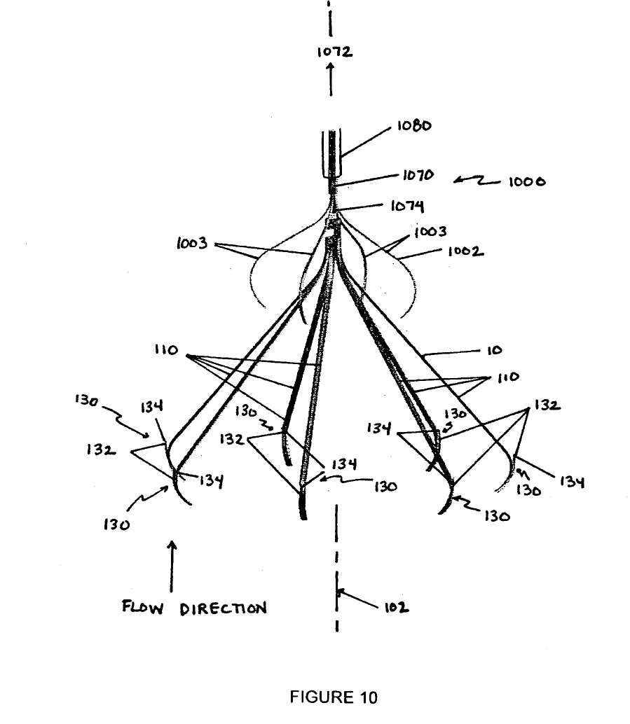

Other embodiments of the present invention include an implant device with an attached tether. The tether permits a temporary implant device to be removed by drawing the implant device out of the body with the tether. However, the tether is detachable from the implant device to convert the implant device into an implant device that may remain indefinitely in the passageway. Again using blood clot filters to demonstrate this aspect of the present invention, FIG. 10 illustrates a tethered filter 1000 which includes the blood clot filter 10 as described with respect to FIG. 1 above. It is understood, however, that the tethered device is not limited to the use of this specific filter. A tether, or micro-tether, 1070, is attached to the blood clot filter 10. The tether 1070 has a proximal end 1072 and a distal end 1074. Thus, the distal end 1072 is located upstream of the proximal end 1074. The distal end 1072 is attached to the filter 10. In addition, the filter is operable at the proximal end 1074 to move the filter 10 within the blood vessel, for instance to move the implant device in the flow direction to attach the filter to the wall of the blood vessel with tissue clips in the manner described previously.

In operation, the tethered filter 1000 is delivered and removed through an introducer sheath 1080 as shown in FIG. 10. The sheath 1080 is a temporary working channel that guides the tethered filter 1000 to the correct position for deployment. Upon positioning the filter, the introducer sheath is retracted allowing the filter legs 110 to expand and come in direct contact with the vessel wall, as described with reference to FIG. 1. It is noted here, but discussed in more detail hereinbelow, that a centering mechanism 1002 is attached to the tether filter 1070 for centering the filter 10 within the wall of the blood vessel upon deployment.

The filter 100 is set by gently pulling on the tether with the filter legs expanded. This motion forces the clip legs 132 to penetrate vessel tissue with their penetrating ends 134, as described previously. The filter 10 is held in place temporarily or permanently with tissue clips 130, and thus, the filter 10 does not rely on column strength of a tether to resist migration. Once the filter 10 is stably and securely deployed with the tissue clips 130, the introducer sheath is removed by fully retracting the sheath over the tether 1070. The tether 1070 is then either externalized at the neck and taped to the chest of the patient or preferably subcutaneously tunneled and coiled in a pocket on the chest or shoulder. In either case, the physician has easy access to the tether if the clinical conditions allow for device removal or device conversion.

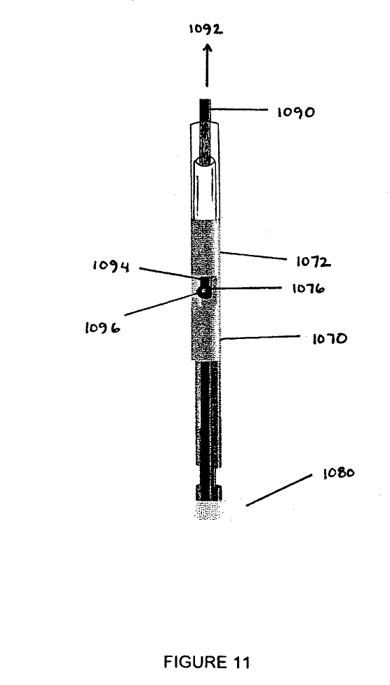

The filter 10 may be removed over the tether 1070 by first recovering the end of the tether 1070 from the subcutaneous pocket and then, as shown in FIG. 11, attaching a extension wire 1090 to the proximal end 1072 of the tether 1070 to an extended length, as referred to as an “exchange length.” The extension wire 1090 also has a proximal end 1092 and a distal end 1094. The distal end 1094 of the extension wire 1090 is attached to the proximal end 1072 of the tether, while the extension wire 1090 is operable from the proximal end 1092. The tether 1070 is preferably extended with the extension wire 1090 so that an introducer sheath 1080 can be guided over the tether 1070 while a physician has control of the proximal end 1092 of the system. As further shown in FIG. 11, the extension wire 1090 may have a protrusion, or ball, 1096 at the distal end 1094. The tether 1070 is extended by feeding the extension wire 1090 with the protrusion 1096 on the end through an external slot 1076 located at the proximal end 1072 of the tether 1070. The extension wire 1090 is fed through the slot 1076 and out the proximal end 1072 of the tether until the protrusion 1096 of the extension wire 1090 prevents further progress. The sheath 1080 is then guided over the tether 1070 to the location of the filter 10 in the blood vessel, and then is further guided over the filter 10 to collapse the filter 10. With the filter 10 collapsed in the sheath 1080, the sheath 1080, the filter 10, and the tether 1070 are withdrawn completely from the blood vessel.

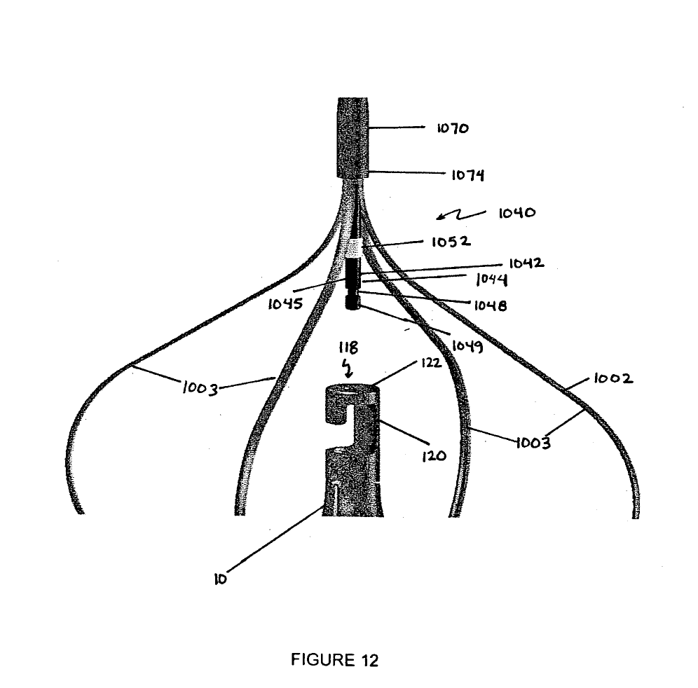

As mentioned above, the tethered filter 1000 enables conversion of a temporary filter to a filter that may remain in the passageway indefinitely. For instance, if the indicated time for temporary use of the filter 10 has elapsed (e.g. 3-6 weeks) and a clot is trapped within the clot capturing basket 105 at the apex 118 of the filter 10, the filter can be converted to an optional configuration, which may remain permanently or be removed at some later time beyond the initial indicated time. As shown in FIG. 12, filter conversion is achieved by recovering the end of the tether 1070 from the subcutaneous pocket and then operating a release mechanism 1040 to release the filter from the tether. The tether 1070 is then pulled from the patient. If necessary, a short introducer sheath can be placed in the neck to facilitate tether removal.

The operation of the release mechanism 1040 is similar to the operation of the control mechanism 140 of FIG. 3. The release mechanism 1040 releasably connects the tether 1070 to the filter cap 120 with the aperture 122 with an aperture width, similar to the control mechanism shown with reference to FIG. 3. The release mechanism 1040 is positioned at the distal end 1074 of the tether 1070. The release mechanism 1040 includes a tube section 1042 extending from the distal end 1072 of the tether 1070. The tube section 1042 has an extended end 1044 that engages the filter 10. The extended end 1044 is provided with a plurality of spaced, longitudinal slits 1045. A control wire 1048, operable from the proximal end 1072 of the tether 1070, extends from the distal end 1074 of the tether 1070, and passes through the tube section 1042. The control wire 1048 has a nodule, or bulb-like structure, 1049 that can move to engage the extended end 1044 of the tube section 1042. Both the distal end 1044 and the nodule 1049 are dimensioned to pass through the aperture 122 when the nodule 1049 does not engage the extended end 1044. In this case, the extended end 1044 and the nodule 1049 each have a width less that the aperture width. A bushing, or stopping mechanism, 1052 secured to the outer surface of the tube section 1042 limits the distance that the tube section 1042 can extend through the filter cap 120.

In the operation of the release mechanism 1040, the blood clot filter 10 is attached to the tether 1070 by passing the tube section 1042 and nodule 1049 through the center aperture 122 in the filter cap 120 until the bushing 1052 engages the filter cap 120. At this point, the slits 1045 at the distal end 144 extend beyond the edge of the filter cap 120. When the nodule 1049 of the control wire 1048 is pulled back toward the proximal end 1072 of the tether 1070, the nodule 1049 moves against and expands the extended end 1044 of the tube section 1042 in the area of the slits 1045. As a result, the tube section 1042 expands outwardly, or radially, at the longitudinal slits 1045 to an expanded width greater than the width of the aperture. Once the extended end 1044 of tube section 1042 is expanded, the filter cap 120 is then positioned, or held, between the bushing 1052 and the expanded end 1044, and the tether 1070 can be used to move the filter 10 longitudinally both in the upstream and flow directions. The control wire 1048 can be attached to a spring in a handle attached to the proximal end 1072 of the tether to hold the nodule 1049 in engagement with the extended end 1044.

The elongate tube 1042 may be formed of nitinol or similar material to facilitate this expansion while permitting the extended end 1044 to return to the non-expanded width when the nodule 1049 is moved from engagement. To release the tether 1070, the release mechanism 1040 from the filter 10, the control wire 1048 is pushed forward to free the nodule 1049 from engagement with the longitudinal slits 1045 at the extended end 1044. As a result, the elongate tube 1042 at the area of the longitudinal slits 1045 is allowed to return to its non-expanded width which is less than the width of the aperture. Accordingly, the nodule 1049 and the tube section 140 can be passed or withdrawn through the aperture 122 of the filter cap 120. Thus, the tether 1070 can be withdrawn from the passageway, leaving the filter 10 in the passageway. If-necessary, the filter 10 can then be later removed from the passageway with a snare, capturing cone, or like device.

Accordingly, attaching a tether to the blood clot filters described previously: 1) addresses the need for prophylactic and temporary placement of vena cava filters in a targeted patient population, 2) provides temporary or permanent protection, from pulmonary embolism, and 3) provides the assurance that the apex of the filter is accessible upon retrieval of temporarily placed filters. The filter can be placed into patients who are at temporary risk of PE using the device placement techniques described above. Although the filter has a tether, the filter is not necessarily a temporary filter, because the filter can be converted into an optional configuration at any time for longer term use by releasing the tether from the filter. Thus, the filter may remain permanently or be removed at a later time.

A centering mechanism is also employed with the tethered filter 1000 of FIG. 10. Unlike the centering mechanism 82 which is attached to the filter 80 in FIG. 8, the centering mechanism 1002 is attached to the tether 1070 and not the filter 10. As illustrated in FIGS. 10 and 12, the centering mechanism 1002 is positioned at the distal end 1074 of the tether 1074, so that the centering mechanism 1002 is proximate to the filter 10. In particular, the centering mechanism 1002 spaces the apex 118 away from the wall of the blood vessel. With the apex 118 centered, the filter 10 is oriented properly for the capture of blood clots traveling through the vessel. Moreover, the filter cap 120 at the apex 118 is less likely to become incorporated with the tissue of the vessel wall creating a risk that the vessel wall could be damaged, especially when the filter 10 is removed. The centering mechanism 1002 has elongate centering legs 1003 that are formed of a flexible material which permits them to be flexibly bendable toward the central toward the central longitudinal axis 102 of the filter 10. Thus, the centering mechanism 1002 is collapsible and expandable. Accordingly, the centering mechanism 1002 is deployed with the filter 10 by collapsing the centering mechanism 1002 and the filter 10 inside the sheath 1080, and guiding the sheath 1080 along the blood vessel until the filter 10 reaches the desired location in the blood vessel. The sheath 1080 is then retracted from the centering mechanism 1080 and the filter 10 to allow the filter 10 and then the centering mechanism 1080 to expand into contact with the wall. Because the centering mechanism 1002 is attached to the tether 1070, it must also be removed if the tether 1070 is withdrawn from the blood vessel, with or without the filter 10. In order to withdraw the centering mechanism 1002 from the blood vessel; the sheath 1080 is guided along the tether 1070 over the centering mechanism 1002 to collapse the centering mechanism 1002 within the sheath 1080 which together with the tether is then guided out of the blood vessel. If the filter 10 is also being removed from the blood vessel, the sheath 1080 would also collapse the filter 10 with the centering mechanism 1002 for removal in the sheath 1080.

In general, embodiments of the present invention may include a centering mechanism operably attached to the implant device proximate to a part of the implant device that should be kept near the center of the body passageway. The centering mechanism includes at least one extension extending outwardly from the centered part into contact against the interior surface of the passageway wall in order to space the wall away from the centered part. Thus, the implant device with the centering mechanism can be guided to a desired location in the passageway. The centering mechanism then keeps the centered part of the implant device near the center of the passageway while the implant device is anchored in place with an attachment mechanism. The centering mechanism may be collapsible for positioning and deployment and expandable to engage the wall of the passageway.

As shown in the FIGS. 8 and 10, the centering mechanism may include a plurality of legs, each having an end extending outwardly in a plurality of directions from the centered part of the implant device into contact against the interior surface of the wall of the passageway. In particular, the legs are arranged evenly in a circular arrangement to space the wall from the centered part of the implant device in all directions by a distance at least equal to the radius of the circle. Although the centering mechanism may be connected directly to the implant device, the centering mechanism may act indirectly to center the implant device, as demonstrated by the embodiment of FIG. 10 where the centering mechanism 1002 is actually attached to the tether 1070.

While the approaches for deployment and removal of implant devices described herein may refer to the use of tissue clips, the approaches are not limited to the use of these particular anchoring mechanisms. The implant devices can be anchored with other anchoring mechanisms, such as hooks that bend and straighten in response to withdrawal forces, as disclosed by U.S. Pat. Nos. 6,007,558 and 6,258,026 to Ravenscroft et al., which are entirely incorporated herein by reference.

While various embodiments in accordance with the present invention have been shown and described, it is understood that the invention is not limited thereto. The present invention may be changed, modified and further applied by those skilled in the art. Therefore, this invention is not limited to the detail shown and described previously, but also includes all such changes and modifications.

What we claim is:

1. An implant device, comprising:
a clip for removably attaching the implant device to a wall of a body passageway, the clip comprising:
a contact portion for contacting the wall of the passageway; and
a clip leg with a sharp tip for engaging the wall of the passageway, the clip leg being positioned on the contact portion and extending away from the contact portion at an angle, the contact portion and the clip leg defining a space for receiving at least apart of the wall of the body passageway,
wherein the contact portion and the clip leg resist an increase in the space between the contact portion and the clip leg.

2. The implant device according to claim 1, wherein the clip leg is substantially straight and substantially rigid.

3. The implant device according to claim 1, further comprising a plurality of elongate legs extending away from a longitudinal axis of the implant device at an angle, each of the plurality of elongate legs having a contact portion, wherein a clip leg is positioned at the contact portion of each of the plurality of elongate legs and extends at an angle from the contact portion.

4. The implant device according to claim 3, further comprising an apex, wherein each of the plurality of elongate legs has a connecting end connected at the apex.

5. The implant device according to claim 4, wherein each of the plurality of legs has a curved end opposite the connecting end, the curved end curving inwardly toward the longitudinal axis.

6. The implant device according to claim 5, wherein the plurality of elongate legs is collapsible for guiding the implant device to a position in the passageway and expandable for removable attachment to the wall of the passageway.

7. The implant device according to claim 6, wherein each of the plurality of elongate legs is flexibly bendable toward the longitudinal axis to make the plurality of elongate legs collapsible.

8. The implant device according to claim 3, wherein the implant device is a blood clot filter with a clot-capturing basket formed by the plurality of elongate legs.

9. The implant device according to claim 8, wherein the clot-capturing basket is formed by adjacent capturing ends of the plurality of elongate legs, the capturing ends not being joined to one another.

10. The implant device according to claim 9, wherein the clot-capturing basket is formed by overlapping of the elongate legs.

11. The implant device according to claim 9, further comprising a centering mechanism to keep the clot-capturing basket centered in the passageway.

12. The implant device according to claim 11, wherein the plurality of elongate legs are connected together at connected ends opposite the capturing ends, and wherein the centering mechanism is attached proximate to the connected ends and comprises a plurality of centering legs extending at an angle from the longitudinal axis of the implant device.

13. The implant device according to claim 12, wherein each centering leg has a centering clip for receiving at least a part of the wall of the body passageway, each centering clip being defined by each centering leg and a centering clip leg positioned on the centering leg, the centering clip leg extending at an angle from the centering leg, wherein the centering leg and the centering clip leg resist movement of the centering clip leg away from the centering leg.

14. The implant device according to claim 1, further comprising an aperture with an aperture width adapted to receive a control mechanism.

15. A system for attaching the implant device according to claim 14 to a wall of a body passageway, wherein the control mechanism comprises:
an elongate tube with a proximal end, and a distal end having a plurality of longitudinal slits;
a control wire that passes through the elongate tube; and
a nodule connected to the control wire at the distal end of the tube, the nodule being movable with operation of the control wire to engage the longitudinal slits of the tube,
wherein the control mechanism engages the implant device when the longitudinal slits at the distal end of the tube are passed through the aperture and the nodule engages the longitudinal slits, causing the tube, to expand outwardly at the longitudinal slits to an expanded width greater than the aperture width, and the control mechanism is releasable from the implant device when the longitudinal slits of the tube are free from engagement by the nodule and the tube has a non-expanded width less than the aperture width, allowing the tube section to pass through the aperture.

16. The system according to claim 15, further comprising a stopping mechanism with a width greater than the aperture width, the stopping mechanism being positioned on the elongate tube and spaced from the distal end of the tube so that the aperture is held between the distal end and the stopping mechanism when the control mechanism engages the implant device.

17. A system for controlling the implant device according to claim 1, the system comprising:
a tether with a distal end and a proximal end, the tether being removably attached to the implant device at the distal end.

18. The system according to claim 17, further comprising an extension wire attached to the proximal end of the tether to extend the tether and to guide the sheath over the implant device.

19. The system according to claim 18, wherein the extension wire has a protrusion at the end of the extension wire that engages a slot at the proximal end of the tether.

20. A method for attaching the implant device according to claim 3 to a wall of a body passageway, the method comprising moving the implant device along the passageway in a first direction, thereby causing movement of the clip legs on the plurality of elongate legs into engagement with the wall of the passageway so that at least a part of the wall is received in the space between the elongate legs and the clip legs.

21. The method according to claim 20, wherein the implant device is a blood-clot filter and moving the implant device along the passageway in a first direction comprises moving the implant device along a blood vessel in a blood flow direction.

22. A method for removing the implant device according to claim 20 from attachment from the wall of a body passageway, the method comprising moving the implant device along the passageway in a second direction opposite the first direction, thereby causing movement of the clip legs on the plurality of elongate legs from engagement with the wall and removal of the wall from the space between the elongate legs and the clip legs.

23. The method according to claim 21, wherein the implant device is a blood-clot filter and moving the implant device along the passageway in the first direction comprises moving the implant device along the blood vessel against the blood flow direction.

24. A method for positioning the implant device according to claim 6 at a location in the passageway, the method comprising:
positioning the implant within a retractable sheath;
guiding the retractable sheath containing the implant device, the sheath keeping the implant device collapsed during movement through the passageway; and
retracting the sheath from the implant device to allow the implant device to expand into attachment with the wall of the passageway at the location.

25. A method for removably attaching the implant device to a wall of a body passageway, comprising:
providing a clip on the implant device, the clip comprising:
a contact portion for contacting the Wall of the passageway; and
a clip leg with a sharp tip for engaging the wall of the passageway, the contact portion and the clip leg defining a space therebetween;
positioning the implant device at a location in the passageway; and
moving the implant device in a first direction to cause the clip leg to engage the wall, and a part of the wall to be clamped in the space between the contact portion and the clip leg.

26. The method according to claim 25, wherein positioning the implant device in the passageway further comprises:
positioning the implant device within a retractable sheath;
guiding the retractable sheath containing the implant device to the location in the passageway, the sheath keeping the clip from engaging the wall of the passageway; and
retracting the sheath from the implant device to position the clip leg against the wall of the passageway at the location.

27. The method according to claim 25, further comprising moving the implant device along the passageway in a second direction opposite the first direction to cause the clip leg to disengage the wall, and the part of the wall to be removed from the space between the contact portion and the clip leg.

28. A system for deploying an implant device to be attached to a wall of a body passageway, the system comprising:
an implant device with an attachment mechanism to attach the implant device to a wall in the passageway, the implant device having an aperture with an aperture width;
a tether with a distal end and a proximal end, the proximal end being operable to control the body; and
a release mechanism adapted to releasably connect the tether to the attachment portion of the implant device.

29. The system of claim 28, wherein the release mechanism comprises:
a tube section extending from the distal end of the tether and having a plurality of longitudinal slits at an extended end of the tube section; and
a control wire, operable from the proximal end of the tether, extending from the distal end of the tether and passing through the tube section, the control wire having a nodule movable to engage the extended end of the tube section, causing the tube section to expand outwardly to an expanded width greater than the aperture width due to the plurality of slits,
wherein the tether is connected to the implant device when the tube section is passed through the aperture and the nodule engages the extended end of the tube section, and the tether is released from the implant device when the extended end of the tube section is free from engagement with the nodule and the tube section has a non-expanded width less than the aperture width, thereby allowing the tube section to pass through the aperture.

30. The system according to claim 29, further comprising a stopping mechanism with a width greater than the aperture width, the stopping mechanism being positioned on the tube section and spaced from the extended end of the tube section so that the aperture is held between the distal end and the stopping mechanism when the tether is connected to the implant device.

31. The system according to claim 28, wherein the attachment mechanism comprises a clip on the implant device including a contact portion for contacting the wall of the passageway, and a clip leg with a sharp tip for engaging the wall of the passageway, the contact portion and the clip leg defining a space therebetween.

32. The system according to claim 28, wherein the attachment mechanism comprises a hook.

33. A method for connecting the tether to the implant device in the system according to claim 29, the method comprising:
passing the tube section through the aperture; and
moving, with the control wire, the nodule into engagement with the extended end of the tube section to cause the tube section to expand outwardly to the expanded width.

34. A method for releasing the tether from the implant device in the system according to claim 29, the method comprising:
moving, with the control wire, the nodule from engagement with the extended end of the tube section to cause the tube section to shrink to the non-expanded width; and
withdrawing the tube section from the aperture.

35. A method for deploying an implant device in a body passageway, the implant device being collapsible for positioning in the passageway and expandable for removable attachment to a wall of the passageway, the method comprising:
attaching a tether to the implant device, the tether having a distal end and a proximal end and being attached to the implant device at the distal end;
guiding the implant device in a retractable sheath to a location in the passageway, the sheath keeping the implant device collapsed during movement through the passageway; and
retracting the sheath from the implant device along the tether, thereby allowing the implant device to expand into attachment with the wall of the passageway at the location.

36. The method according to claim 35, further comprising:
detaching the tether from the implant device by operating the proximal end of the tether and activating a release mechanism to release the implant device from the tether; and
withdrawing the tether from the passageway.

37. The method according to claim 36, further comprising removing the implant device, after detaching the tether, with a snare or capturing cone.

38. The method according to claim 37, further comprising removing the implant device from the passageway, removing the implant device comprising:
guiding the sheath over the tether to the location of the implant device in the passageway and over the implant device to collapse the implant device; and
withdrawing the sheath, implant device, and tether from the passageway.

39. The method according to claim 38, further comprising removing the implant device from the passageway by extending the tether with an extension wire to the tether in order to guide the sheath over the tether and the implant device, whereby the implant device is collapsed and removable from the passageway.

40. The method according to claim 39, wherein extending the tether with an extension wire comprises guiding a protrusion at an end of the extension wire into contact with the tether and into engagement with a slot at the proximal end of the tether.

41. The method of claim 36, further comprising storing the proximal end of the tether subcutaneously.

42. The method according to claim 35, wherein guiding the implant device further comprises centering a part of the implant device near the center of the passageway with a centering mechanism attached to the tether proximate to the implant device.

43. The method according to claim 42, further comprising:
guiding the retractable sheath containing the centering mechanism with the implant device, the sheath keeping the centering mechanism collapsed during movement through the passageway, and
retracting the sheath from the centering mechanism to allow the centering mechanism to expand into centering contact with the wall of the passageway at the location,
wherein the centering mechanism is collapsible for deployment and expandable to engage the wall of the passageway.

44. A system for deploying an implant device in a body passageway, the implant device being collapsible for positioning in the passageway and expandable for removable attachment to a wall of the passageway, the system comprising:
a tether attachable to the implant device, the tether having a distal end and a proximal end, and being attached to the implant device at the distal end; and
a flexible, retractable sheath with an elongate channel, the tether extending through the sheath, the implant device being collapsible within the channel and expandable when the sheath is retracted along the tether from the implant device.

45. The system according to claim 44, wherein the proximal end of the tether is operable to activate a release mechanism to detach the tether from the implant device.

46. The system according to claim 44, further comprising a snare or capturing cone for removing the implant device.

47. The system according to claim 44, wherein the sheath is guidable over the tether and over the implant device to collapse the implant device.

48. The system according to claim 44, further comprising an extension wire attachable to the tether, the sheath being guidable over the extension wire and tether to collapse the implant device.

49. The system according to claim 48, further comprising:
a slot at the proximal end of the tether; and
a protrusion at an end of the extension wire adapted to engage the slot of the tether in order to attach the extension wire to the tether.

50. The system according to claim 44, further comprising a centering mechanism attachable to the tether proximate to the implant device to center a part of the implant device.

51. The system according to claim 50, wherein the centering mechanism is collapsible within the channel of the retractable sheath and expandable when the sheath is retracted along the tether from the implant device.

52. The system according to claim 44, wherein the centering mechanism comprises a longitudinal axis and a plurality of centering legs, each of the plurality of centering legs extending along the longitudinal axis and away from the longitudinal axis.

53. A method for deploying an implant device in a body passageway, the implant device having an attachment mechanism to attach the implant device to a wall in the passageway, the method comprising:
attaching a centering mechanism to the implant device proximate to a centered part of the implant device to be kept near the center of the passageway, the centering mechanism comprising at least one extension extending outwardly from the centered part of the implant device for spacing the wall from the centered part;
guiding the implant device with the centering mechanism to a location in the passageway; and
attaching the implant device to the wall with the attachment mechanism while the centering mechanism keeps the centered part of the implant device near the center of the passageway.

54. The method according to claim 53, wherein the centering mechanism comprises a plurality of legs, each of the plurality of legs having an end extending outwardly in a plurality of directions from the centered part of the implant device, into contact against the interior surface of the wall of the passageway.

55. The method according to claim 53, wherein the plurality of legs are evenly spaced in a circular arrangement.

56. The method according to claim 53, wherein the centering mechanism is a part of a tether attached to the implant device.

57. The method according to claim 53, wherein the centering mechanism is collapsible for deployment and expandable to engage the wall of the passageway.

58. The method according to claim 53, wherein the implant device is collapsible for positioning in the passageway and expandable for attachment to a wall of the passageway, and wherein guiding the implant device with the centering mechanism comprises moving the retractable sheath containing the implant device with the centering mechanism, the sheath keeping the implant device and the centering mechanism collapsed during movement through the passageway, and wherein attaching the implant device to the wall with the attachment mechanism comprises retracting the sheath from the implant device to permit the implant device and the centering mechanism to expand into contact with the wall of the passageway.
